SEQUENCE LISTING (14 sequences)

SEQ ID NO: 1 - DNA, 29 bp, synthetic construct
  source 1..29: mol_type = other DNA
gggaatcacatcagtaactgagtgacttc

SEQ ID NO: 2 - DNA, 2862 bp, synthetic construct
  source 1..2862: mol_type = other DNA
atgggcaaaaaaatccatgcacgtgatctgcgtgaacagcgtaaaaccgatcgtaccgaaaaatttgcagaccagaacaaaaaacgtgaagcagaacgtgcagttccgaaaaaagatgcagcagttagcgttaaaagcgttagcagcgtgagcagcaaaaaagataatgttaccaaaagcatggcaaaagcagccggtgtgaaaagcgtttttgcagttggtaataccgtttacatgaccagctttggtcgtggtaatgatgcagttctggaacagaaaattgttgataccagccatgaaccgctgaatattgatgatccggcatatcagctgaatgtggttaccatgaatggttatagcgttaccggtcatcgtggtgaaaccgttagcgcagttaccgataatccgctgcgtcgttttaatggtcgtaaaaaagacgaaccggaacagagcgttccgaccgatatgctgtgtctgaaaccgacactggaaaaaaagttttttggcaaagagttcgacgacaacattcatatccagctgatttataacatcctggacatcgaaaaaatcctggcagtttatagcaccaatgcaatttatgccctgaataacatgagcgcagatgaaaacattgagaacagcgacttttttatgaaacgcaccaccgatgaaaccttcgatgatttcgaaaaaaagaaagaaagcaccaacagccgtgaaaaagcagattttgatgccttcgaaaaattcatcggtaattatcgcctggcctattttgcagatgccttttatgtcaacaaaaagaacccgaaaggcaaagccaaaaatgtgctgcgtgaagataaagaactgtatagcgttctgaccctgattggtaaactgcgtcattggtgtgttcatagcgaagaaggtcgcgcagaattttggctgtataaactggatgaactgaaggacgatttcaaaaacgttctggatgtggtttataatcgtccggtggaagaaattaacaaccgctttatcgaaaacaacaaagtgaacattcagattctgggcagcgtgtataaaaacaccgatattgcagaactggtgcgcagctattatgaatttctgatcaccaaaaaatacaaaaacatgggctttagcatcaaaaaactgcgtgaaagcatgctggaaggtaaaggttatgccgataaagaatatgatagcgtgcgcaacaaactgtatcagatgaccgattttattctgtacaccggctatatcaacgaagatagcgatcgtgcagatgatctggttaataccctgcgtagcagcctgaaagaagatgataaaaccaccgtgtattgcaaagaggcagattacctttggaaaaagtatcgcgaaagcattcgtgaagttgccgatgcactggatggtgacaatattaagaaactgagcaaaagcaacatcgagatccaagaggataaactgcgcaaatgctttattagctatgcagatagcgttagcgaatttaccaaactgatttatctgctgacccgttttctgagcggtaaagaaatcaacgatctggtgaccacactgatcaacaaatttgataacattcgcagctttctggaaatcatggatgaattaggtctggatcgtacctttaccgcagaatatagcttttttgaaggcagcaccaaatatctggccgaactggtggaactgaatagctttgttaaatcctgcagcttcgatattaatgccaaacgtaccatgtatcgtgatgccctggatattctgggtattgaatcagataaaaccgaagaggatatcgagaaaatgatcgacaatatcctgcagattgatgccaacggtgataaaaagctgaaaaaaaacaatggcctgcgcaactttattgccagcaatgttattgatagcaaccgcttcaaatacctggtgcgttatggtaacccgaagaaaattcgtgaaaccgcaaaatgtaaaccggcagttcgttttgtgctgaatgaaattccggatgcacagatcgaacgttattatgaagcatgttgtccgaaaaataccgcactgtgtagcgcaaataaacgtcgtgaaaaactggcagatatgatcgccgaaatcaaattcgaaaacttttccgatgcaggcaactatcagaaagcaaatgtgaccagccgtaccagcgaagcagaaattaaacgtaaaaaccaggccattattcgcctgtatctgaccgttatgtatatcatgctgaaaaacctggtgaatgtgaatgcccgttatgtgattgcatttcattgtgttgaacgcgacacaaaactgtatgccgaaagcggtctggaagtgggtaatattgaaaagaataaaaccaacctgaccatggccgttatgggtgttaaactggaaaatggcattatcaaaaccgagttcgataagagctttgcagaaaatgcagcaaatcgttatctgcgtaatgcgcgttggtataagctgattctggataatctgaagaaaagcgaacgtgccgttgtgaatgaatttcgcaataccgtgtgtcatctgaatgccattcgcaacattaacatcaacattaaagagatcaaagaggtcgagaactatttcgccctgtatcattatctgatccagaaacatctggaaaaccgcttcgcagacaaaaaagttgaacgtgataccggtgatttcatcagcaaactggaagaacataaaacctactgcaaagacttcgtgaaagcctattgtaccccgtttggttataatctggttcgctataaaaacctgacgatcgatggtctgttcgacaaaaactatccgggtaaagatgatagcgacgagcagaaa

SEQ ID NO: 3 - DNA, 3477 bp, synthetic construct
  source 1..3477: mol_type = other DNA
atgaaagtgacgaaggtaggaggcatttcgcataagaagtacacgtccgaaggccgcttagtgaagtcagaatcggaagaaaatcgcacagacgaacgtctgtcggcgttgcttaatatgcgccttgacatgtatatcaagaatcccagcagcacggaaaccaaggaaaatcaaaaacgcattgggaaattaaagaaattcttctcaaacaaaatggtctatcttaaagacaataccttgagtttgaagaatgggaaaaaggagaacattgatcgtgagtattctgagactgacatccttgagagcgatgtccgtgacaagaaaaacttcgccgtgttgaaaaagatctatctgaatgaaaacgtgaactcggaggaattggaagtttttcgtaacgacattaagaagaaactgaacaaaatcaacagcctgaagtactcatttgaaaagaataaggcgaattatcaaaagattaatgagaataacatcgagaaggttgaaggtaagtcaaagcgtaacattatttacgattattatcgtgagtcagcgaaacgtgacgcttatgtaagcaatgtgaaagaagcctttgataagctttacaaggaagaggacattgcaaaacttgttcttgaaattgagaaccttacgaagttagagaaatacaagattcgcgagttctaccacgaaattattggacgtaagaatgacaaggaaaactttgcaaaaatcatctacgaagaaatccagaatgttaataacatgaaagagttgatcgagaaggtaccggacatgagtgagttgaaaaagagccaagtattttacaagtattacttagacaaagaagagttgaacgacaagaacatcaaatacgcgttttgtcatttcgtggaaatcgaaatgagtcagttgctgaagaactacgtatataagcgcttaagtaatatctcgaatgacaaaattaagcgtatctttgaataccagaacttgaaaaaattgatcgaaaataagctgttaaacaaacttgacacgtacgtccgtaattgtggaaagtataattattatttgcaagacggcgaaattgccacttcagatttcatcgcccgcaaccgtcagaatgaagcgtttcttcgcaacatcattggggtgtcatctgtggcctacttttctcttcgcaacattcttgaaacggagaacgagaatgatattactgggcgtatgcgcggcaaaacagttaagaacaataaaggtgaagagaagtacgtgtccggagaagttgataagatctataatgaaaataagaagaacgaggttaaggagaacttaaaaatgttctattcgtacgatttcaatatggacaacaagaatgaaatcgaagatttcttcgccaacatcgacgaggcgatttcttccatccgtcacggtattgtccacttcaacttggaattagaaggtaaggatatctttgcgttcaagaacattgcgccatccgaaatctcaaagaagatgtttcagaatgagattaacgagaaaaaactgaaattgaagatctttcgtcaactgaactctgccaacgtgttccgctatctcgaaaagtataaaattctgaattaccttaaacgtacacgcttcgagtttgtcaataaaaatatcccattcgtcccgtctttcaccaaattatattcgcgcattgatgacctgaagaatagtcttgggatttactggaaaactccgaaaacaaacgacgacaataagactaaggagattattgatgcccaaatctatttgcttaaaaacatctattacggggagttcctgaattatttcatgtcgaacaatggtaatttctttgagatttctaaagaaatcatcgaattgaacaagaacgataaacgcaacttaaagactgggttttacaagctgcaaaagtttgaagacatccaggagaagattccaaaggaatacttggcgaatatccagtccctgtacatgattaatgccggtaatcaggacgaagaagaaaaggacacttatattgatttcattcaaaagatcttcttaaagggatttatgacgtatcttgctaataacggtcgtttaagtctgatttacatcggctcggatgaagaaacaaatacgtcattagcagaaaagaagcaagagtttgacaagttcttgaagaagtacgagcagaacaataatatcaagatcccctatgagatcaatgaattcctgcgtgagatcaaactgggaaacatcctgaagtatactgagcgtttaaacatgttctaccttatcttaaagcttttgaatcacaaggagctgacaaatctgaagggtagtcttgaaaaatatcagtctgccaataaggaagaagcgttctctgaccaattggagttaattaacctgcttaaccttgacaacaaccgcgtgacggaagacttcgaattagaggccgacgagattggaaaatttcttgatttcaatggcaacaaagttaaggataacaaggaactgaaaaagttcgatacaaacaagatctactttgacggcgagaacattatcaaacaccgtgccttctacaatattaagaaatatggcatgttaaacttactggagaaaattgccgacaaggctggatacaagatctcgatcgaagagctgaagaaatactccaataaaaagaatgagatcgagaagaaccataagatgcaggaaaatctgcaccgcaaatacgctcgtccccgtaaagacgagaagtttacagatgaggactatgaaagttacaagcaagctattgagaatattgaggagtacacccaccttaagaacaaggtagaattcaatgagctgaatttactgcagggcctgttgctgcgcattttacatcgtttagtcggatatacctcaatttgggaacgcgatctgcgcttccgccttaaaggtgagttcccagaaaaccaatacatcgaagagatcttcaactttgaaaataagaagaacgtgaagtacaaagggggtcagattgtagagaaatacattaaattctacaaggaattacatcaaaatgatgaagttaagatcaacaagtacagttccgcgaatatcaaggtgttgaagcaagaaaagaaggacctttatattcgaaattacatcgcccacttcaattatattcctcacgccgagatctcactgctggaagtccttgaaaatttgcgtaaattgctgtcctacgatcgcaaactgaaaaatgccgtaatgaaatcagtagttgatatccttaaggagtatggttttgtagccacattcaaaatcggggcggacaagaagatcggtattcagacactggagagcgaaaaaatcgtgcatcttaagaatcttaagaagaagaagttaatgactgaccgcaattccgaggaactttgcaaattggtgaagattatgtttgaatacaaaatggaagagaaaaagtctgaaaac

SEQ ID NO: 4 - DNA, 29 bp, synthetic construct
  source 1..29: mol_type = other DNA
acatggtaccacatatatcacgtcaacgt

SEQ ID NO: 5 - DNA, 22 bp, synthetic construct
  source 1..22: mol_type = other DNA
tgaaccaagacgcagtattatt

SEQ ID NO: 6 - DNA, 31 bp, synthetic construct
  source 1..31: mol_type = other DNA
ggaccaccccaaaaatgaaggggactaaaac

SEQ ID NO: 7 - DNA, 402 bp, Escherichia sp.
  source 1..402: mol_type = genomic DNA
atggcaaaattagagactgttactttaggtaacatcgggaaagatggaaaacaaactctggtcctcaatccgcgtggggtaaatcccactaacggcgttgcctcgctttcacaagcgggtgcagttcctgcgctggagaagcgtgttaccgtttcggtatctcagccttctcgcaatcgtaagaactacaaggtccaggttaagatccagaacccgaccgcttgcactgcaaacggttcttgtgacccatccgttactcgccaggcatatgctgacgtgaccttttcgttcacgcagtatagtaccgatgaggaacgagcttttgttcgtacagagcttgctgctctgctcgctagtcctctgctgatcgatgctattgatcagctgaacccagcgtattga

SEQ ID NO: 8 - protein, 133 aa, Escherichia sp.
MAKLETVTLGNIGKDGKQTLVLNPRGVNPTNGVASLSQAGAVPALEKRVTVSVSQPSRNRKNYKVQVKIQNPTACTANGSCDPSVTRQAYADVTFSFTQYSTDEERAFVRTELAALLASPLLIDAIDQLNPAY

SEQ ID NO: 9 - DNA, 396 bp, Acinetobacter phage
  source 1..396: mol_type = genomic DNA
atggcaaataagccaatgcaaccgatcacatctacagcaaataaaattgtgtggagtgatccaactcgtttatcaactacattttcagcaagtctgttacgccaacgtgttaaagttggtatagccgaactgaataatgtttcaggtcaatatgtatctgtttataagcgtcctgcacctaaaccggaaggttgtgcagatgcctgtgtcattatgccgaatgaaaaccaatccattcgcacagtgatttcagggtcagccgaaaacttggctaccttaaaagcagaatgggaaactcacaaacgtaacgttgacacactcttcgcgagcggcaacgccggtttgggtttccttgaccctactgcggctatcgtatcgtctgatactactgcttaa

SEQ ID NO: 10 - protein, 131 aa, Acinetobacter phage
MANKPMQPITSTANKIVWSDPTRLSTTFSASLLRQRVKVGIAELNNVSGQYVSVYKRPAPKPEGCADACVIMPNENQSIRTVISGSAENLATLKAEWETHKRNVDTLFASGNAGLGFLDPTAAIVSSDTTA

SEQ ID NO: 11 - DNA, 3677 bp, synthetic construct
  source 1..3677: mol_type = other DNA
taatacgactcactataggggaatcacatcagtaactgagtgacttcggcgaattaattccggttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctcacctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataatatgctggccaccacccatatgggatccatgggcaaaaaaatccatgcacgtgatctgcgtgaacagcgtaaaaccgatcgtaccgaaaaatttgcagaccagaacaaaaaacgtgaagcagaacgtgcagttccgaaaaaagatgcagcagttagcgttaaaagcgttagcagcgtgagcagcaaaaaagataatgttaccaaaagcatggcaaaagcagccggtgtgaaaagcgtttttgcagttggtaataccgtttacatgaccagctttggtcgtggtaatgatgcagttctggaacagaaaattgttgataccagccatgaaccgctgaatattgatgatccggcatatcagctgaatgtggttaccatgaatggttatagcgttaccggtcatcgtggtgaaaccgttagcgcagttaccgataatccgctgcgtcgttttaatggtcgtaaaaaagacgaaccggaacagagcgttccgaccgatatgctgtgtctgaaaccgacactggaaaaaaagttttttggcaaagagttcgacgacaacattcatatccagctgatttataacatcctggacatcgaaaaaatcctggcagtttatagcaccaatgcaatttatgccctgaataacatgagcgcagatgaaaacattgagaacagcgacttttttatgaaacgcaccaccgatgaaaccttcgatgatttcgaaaaaaagaaagaaagcaccaacagccgtgaaaaagcagattttgatgccttcgaaaaattcatcggtaattatcgcctggcctattttgcagatgccttttatgtcaacaaaaagaacccgaaaggcaaagccaaaaatgtgctgcgtgaagataaagaactgtatagcgttctgaccctgattggtaaactgcgtcattggtgtgttcatagcgaagaaggtcgcgcagaattttggctgtataaactggatgaactgaaggacgatttcaaaaacgttctggatgtggtttataatcgtccggtggaagaaattaacaaccgctttatcgaaaacaacaaagtgaacattcagattctgggcagcgtgtataaaaacaccgatattgcagaactggtgcgcagctattatgaatttctgatcaccaaaaaatacaaaaacatgggctttagcatcaaaaaactgcgtgaaagcatgctggaaggtaaaggttatgccgataaagaatatgatagcgtgcgcaacaaactgtatcagatgaccgattttattctgtacaccggctatatcaacgaagatagcgatcgtgcagatgatctggttaataccctgcgtagcagcctgaaagaagatgataaaaccaccgtgtattgcaaagaggcagattacctttggaaaaagtatcgcgaaagcattcgtgaagttgccgatgcactggatggtgacaatattaagaaactgagcaaaagcaacatcgagatccaagaggataaactgcgcaaatgctttattagctatgcagatagcgttagcgaatttaccaaactgatttatctgctgacccgttttctgagcggtaaagaaatcaacgatctggtgaccacactgatcaacaaatttgataacattcgcagctttctggaaatcatggatgaattaggtctggatcgtacctttaccgcagaatatagcttttttgaaggcagcaccaaatatctggccgaactggtggaactgaatagctttgttaaatcctgcagcttcgatattaatgccaaacgtaccatgtatcgtgatgccctggatattctgggtattgaatcagataaaaccgaagaggatatcgagaaaatgatcgacaatatcctgcagattgatgccaacggtgataaaaagctgaaaaaaaacaatggcctgcgcaactttattgccagcaatgttattgatagcaaccgcttcaaatacctggtgcgttatggtaacccgaagaaaattcgtgaaaccgcaaaatgtaaaccggcagttcgttttgtgctgaatgaaattccggatgcacagatcgaacgttattatgaagcatgttgtccgaaaaataccgcactgtgtagcgcaaataaacgtcgtgaaaaactggcagatatgatcgccgaaatcaaattcgaaaacttttccgatgcaggcaactatcagaaagcaaatgtgaccagccgtaccagcgaagcagaaattaaacgtaaaaaccaggccattattcgcctgtatctgaccgttatgtatatcatgctgaaaaacctggtgaatgtgaatgcccgttatgtgattgcatttcattgtgttgaacgcgacacaaaactgtatgccgaaagcggtctggaagtgggtaatattgaaaagaataaaaccaacctgaccatggccgttatgggtgttaaactggaaaatggcattatcaaaaccgagttcgataagagctttgcagaaaatgcagcaaatcgttatctgcgtaatgcgcgttggtataagctgattctggataatctgaagaaaagcgaacgtgccgttgtgaatgaatttcgcaataccgtgtgtcatctgaatgccattcgcaacattaacatcaacattaaagagatcaaagaggtcgagaactatttcgccctgtatcattatctgatccagaaacatctggaaaaccgcttcgcagacaaaaaagttgaacgtgataccggtgatttcatcagcaaactggaagaacataaaacctactgcaaagacttcgtgaaagcctattgtaccccgtttggttataatctggttcgctataaaaacctgacgatcgatggtctgttcgacaaaaactatccgggtaaagatgatagcgacgagcagaaagaattcgatatcttaattaagctgcaggagctcgtcgacgcggccgcactcgagtgagatctgactgaaaaaaaaaaaaaaaaaaaaaaaaaaaaaagtttaaacactagtccgctgagcaataactagcataaccccttggggcctctaaacgggtcttgaggggttttttgctgaaaggaggaactatatccgggcttcctcgctcactgactcgctgcgctcgg

SEQ ID NO: 12 - DNA, 4292 bp, synthetic construct
  source 1..4292: mol_type = other DNA
taatacgactcactataggggaatcacatcagtaactgagtgacttcggcgaattaattccggttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctcacctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataatatgctggccaccacccatatgggatccatgaaagtgacgaaggtaggaggcatttcgcataagaagtacacgtccgaaggccgcttagtgaagtcagaatcggaagaaaatcgcacagacgaacgtctgtcggcgttgcttaatatgcgccttgacatgtatatcaagaatcccagcagcacggaaaccaaggaaaatcaaaaacgcattgggaaattaaagaaattcttctcaaacaaaatggtctatcttaaagacaataccttgagtttgaagaatgggaaaaaggagaacattgatcgtgagtattctgagactgacatccttgagagcgatgtccgtgacaagaaaaacttcgccgtgttgaaaaagatctatctgaatgaaaacgtgaactcggaggaattggaagtttttcgtaacgacattaagaagaaactgaacaaaatcaacagcctgaagtactcatttgaaaagaataaggcgaattatcaaaagattaatgagaataacatcgagaaggttgaaggtaagtcaaagcgtaacattatttacgattattatcgtgagtcagcgaaacgtgacgcttatgtaagcaatgtgaaagaagcctttgataagctttacaaggaagaggacattgcaaaacttgttcttgaaattgagaaccttacgaagttagagaaatacaagattcgcgagttctaccacgaaattattggacgtaagaatgacaaggaaaactttgcaaaaatcatctacgaagaaatccagaatgttaataacatgaaagagttgatcgagaaggtaccggacatgagtgagttgaaaaagagccaagtattttacaagtattacttagacaaagaagagttgaacgacaagaacatcaaatacgcgttttgtcatttcgtggaaatcgaaatgagtcagttgctgaagaactacgtatataagcgcttaagtaatatctcgaatgacaaaattaagcgtatctttgaataccagaacttgaaaaaattgatcgaaaataagctgttaaacaaacttgacacgtacgtccgtaattgtggaaagtataattattatttgcaagacggcgaaattgccacttcagatttcatcgcccgcaaccgtcagaatgaagcgtttcttcgcaacatcattggggtgtcatctgtggcctacttttctcttcgcaacattcttgaaacggagaacgagaatgatattactgggcgtatgcgcggcaaaacagttaagaacaataaaggtgaagagaagtacgtgtccggagaagttgataagatctataatgaaaataagaagaacgaggttaaggagaacttaaaaatgttctattcgtacgatttcaatatggacaacaagaatgaaatcgaagatttcttcgccaacatcgacgaggcgatttcttccatccgtcacggtattgtccacttcaacttggaattagaaggtaaggatatctttgcgttcaagaacattgcgccatccgaaatctcaaagaagatgtttcagaatgagattaacgagaaaaaactgaaattgaagatctttcgtcaactgaactctgccaacgtgttccgctatctcgaaaagtataaaattctgaattaccttaaacgtacacgcttcgagtttgtcaataaaaatatcccattcgtcccgtctttcaccaaattatattcgcgcattgatgacctgaagaatagtcttgggatttactggaaaactccgaaaacaaacgacgacaataagactaaggagattattgatgcccaaatctatttgcttaaaaacatctattacggggagttcctgaattatttcatgtcgaacaatggtaatttctttgagatttctaaagaaatcatcgaattgaacaagaacgataaacgcaacttaaagactgggttttacaagctgcaaaagtttgaagacatccaggagaagattccaaaggaatacttggcgaatatccagtccctgtacatgattaatgccggtaatcaggacgaagaagaaaaggacacttatattgatttcattcaaaagatcttcttaaagggatttatgacgtatcttgctaataacggtcgtttaagtctgatttacatcggctcggatgaagaaacaaatacgtcattagcagaaaagaagcaagagtttgacaagttcttgaagaagtacgagcagaacaataatatcaagatcccctatgagatcaatgaattcctgcgtgagatcaaactgggaaacatcctgaagtatactgagcgtttaaacatgttctaccttatcttaaagcttttgaatcacaaggagctgacaaatctgaagggtagtcttgaaaaatatcagtctgccaataaggaagaagcgttctctgaccaattggagttaattaacctgcttaaccttgacaacaaccgcgtgacggaagacttcgaattagaggccgacgagattggaaaatttcttgatttcaatggcaacaaagttaaggataacaaggaactgaaaaagttcgatacaaacaagatctactttgacggcgagaacattatcaaacaccgtgccttctacaatattaagaaatatggcatgttaaacttactggagaaaattgccgacaaggctggatacaagatctcgatcgaagagctgaagaaatactccaataaaaagaatgagatcgagaagaaccataagatgcaggaaaatctgcaccgcaaatacgctcgtccccgtaaagacgagaagtttacagatgaggactatgaaagttacaagcaagctattgagaatattgaggagtacacccaccttaagaacaaggtagaattcaatgagctgaatttactgcagggcctgttgctgcgcattttacatcgtttagtcggatatacctcaatttgggaacgcgatctgcgcttccgccttaaaggtgagttcccagaaaaccaatacatcgaagagatcttcaactttgaaaataagaagaacgtgaagtacaaagggggtcagattgtagagaaatacattaaattctacaaggaattacatcaaaatgatgaagttaagatcaacaagtacagttccgcgaatatcaaggtgttgaagcaagaaaagaaggacctttatattcgaaattacatcgcccacttcaattatattcctcacgccgagatctcactgctggaagtccttgaaaatttgcgtaaattgctgtcctacgatcgcaaactgaaaaatgccgtaatgaaatcagtagttgatatccttaaggagtatggttttgtagccacattcaaaatcggggcggacaagaagatcggtattcagacactggagagcgaaaaaatcgtgcatcttaagaatcttaagaagaagaagttaatgactgaccgcaattccgaggaactttgcaaattggtgaagattatgtttgaatacaaaatggaagagaaaaagtctgaaaacgaattcgatatcttaattaagctgcaggagctcgtcgacgcggccgcactcgagtgagatctgactgaaaaaaaaaaaaaaaaaaaaaaaaaaaaaagtttaaacactagtccgctgagcaataactagcataaccccttggggcctctaaacgggtcttgaggggttttttgctgaaaggaggaactatatccgggcttcctcgctcactgactcgctgcgctcgg

SEQ ID NO: 13 - DNA, 167 bp, synthetic construct
  source 1..167: mol_type = other DNA
tctagataatacgactcactataggggaccaccccaaaaatgaaggggactaaaacggatccacatggtaccacatatatcacgtcaacgtcggtaccggatcaggatcaggagggaatcacatcagtaactgagtgacttcgctagttattgctcagcgggaattc

SEQ ID NO: 14 - DNA, 159 bp, synthetic construct
  source 1..159: mol_type = other DNA
tctagataatacgactcactataggggaccaccccaaaaatgaaggggactaaaacggatcctgaaccaagacgcagtattattggtaccggatcaggatcaggagggaatcacatcagtaactgagtgacttcgctagttattgctcagcgggaattc

(19) United States
(12) Patent Application Publication
(10) Pub. No.: US 2024/0197838 A1
(43) Pub. Date: Jun. 20, 2024

(54) mRNA NANOCAPSULE

(71) Applicant: VL Biotech Inc., New Taipei (TW)

(72) Inventor: Chiao-long HSIAO, Taipei (TW)

(21) Appl. No.: 18/429,193

(22) Filed: Jan. 31, 2024

Related U.S. Application Data
(63) Continuation-in-part of application No. 17/528,851, filed on Nov. 17, 2021, now abandoned.
(60) Provisional application No. 63/146,914, filed on Feb. 8, 2021.

(51) Int. Cl. A61K 38/46; A61K 9/51

(52) U.S. Cl. CPC A61K 38/465; A61K 9/5184

(57) ABSTRACT

The present invention provides an mRNA nanocapsule and use thereof, comprising a virus-like particle (VLP) formed by self-assembly of a plurality of capsid proteins (CPs), an mRNA encoding Cas13 protein, and a guide RNA. The mRNA includes a capsid protein binding tag to be encapsidated in the VLP so that the mRNA can stably enter cells and the Cas13 protein can be translated.

### REFERENCE TO AN ELECTRONIC SEQUENCE LISTING

The contents of the electronic sequence listing (Sequence Listing.xml; Size: 28,884 bytes; and Date of Creation: Jan. 31, 2024) are herein incorporated by reference in its entirety.

### FIELD OF THE INVENTION

The present invention relates to a drug delivery technology, and in particular, to a technology of delivering mRNA within self-assembling nanocapsules for drug delivery.

### BACKGROUND OF THE INVENTION

Severe Acute Respiratory Syndrome Coronavirus Type 2 (also known as Novel Coronavirus, SARS-COV-2) is an enveloped single-stranded RNA virus that spreads mainly in the form of droplets such as coughing or sneezing and passes through the human respiratory tract to cause infection, inducing the symptoms such as low fever, weakness, oral and nasal symptoms, dry cough, and gastrointestinal discomfort, etc. The severe special infectious pneumonia (COVID-19) caused by SARS-COV-2 has rapidly caused severe epidemics around the world since the end of 2019, with a total of nearly 200 million people infected and more than 4 million deaths.

As a means of prevention of public health at present, the common strategy against COVID-19 is vaccination worldwide. However, after vaccination, it will take nearly one month to produce enough antibodies against the virus through the immune response. Furthermore, once the number of infected people accumulates in a short period of time, it will put pressure on the production capacity of vaccine manufacturers and the distribution of vaccines in the global community, in addition to destroying the medical systems. Finally, SARS-COV-2 has the characteristics of rapid mutation, and the numerous variants make the efficacy of existing vaccines questionable. Therefore, if drugs that can effectively treat and prevent COVID-19 can be developed, they will provide another weapon for humans fighting against the epidemic.

SARS-COV-2, similar to common Coronaviruses, is a large and enveloped spherical single-stranded RNA virus, namely, its genetic material is ribonucleic acid (RNA). Therefore, if RNA of the SARS-CoV-2 can be destroyed, the in vivo replication and proliferation of SARS-COV-2 can be prevented, thereby achieving the effect of treating and preventing COVID-19.

The CRISPR/Cas system is an acquired immune system found in most bacteria. It comprises Clustered Regularly Interspaced Short Palindromic Repeats (CRISPR) and CRISPR-associated proteins (hereinafter referred to as Cas protein). Cas13 protein is an RNA nuclease that can bind with guide RNA to detect specific RNA sequences and cleave them, that is, this CRISPR/Cas system can be used to destroy RNA of the SARS-COV-2. However, it is a problem how to transfer this system to human cells safely and completely to achieve the above effect effectively. At present, the Cas13 system has been proved to be effective against SARS-COV-2 and influenza virus in challenge test in animal models. However, considering the tendency of disintegration due to unstable nature of mRNA, previous experiments needed to adopt transfection methods that are toxic to cells.

Moreover, Cas13 mRNA should be prepared by in vitro transcription in the past, so it is not conducive to actual clinical uses. Besides, conventional techniques for mRNA delivery often involve lipid nanoparticles; however, it is hard for the lipid nanoparticles to provide a stable biological environment, and there are also issues with potential immunogenicity and inefficiency in encapsulating and delivering larger molecules such as mRNA encoding the Cas protein. Therefore, mRNA delivery technology for large molecules is an urgent era for medical acquisition.

### SUMMARY OF THE INVENTION

To solve the foregoing problems, the invention provides an mRNA nanocapsule, which makes mRNA encoding a Cas13 protein to be bound to virus-like particle (VLP) and encapsulated in the VLP to form a nanocapsule for drug delivery.

Aiming to the above goal, the present invention provides an mRNA nanocapsule, comprising: a plurality of capsid proteins (CPs), self-assembling into a virus-like particle (VLP), wherein each of the plurality of CPs comprises an RNA-binding motif; at least one first RNA molecule, comprising an mRNA encoding a Cas13 protein and a capsid protein binding tag linked with the mRNA, wherein the capsid protein binding tag recognizing and binding to the RNA-binding motif of one of the plurality of CPs, and the capsid protein binding tag is encoded by a VLP recognition sequence as denoted by SEQ ID NO: 1; and at least one second RNA molecule, encoding by a nucleotide sequence comprising a targeting sequence reverse and complementary to a targeted site, a Cas13 protein recognition sequence, and the VLP recognition sequence as denoted by SEQ ID NO: 1, wherein the VLP recognition sequence encodes the capsid protein binding tag recognizing and binding to the RNA-binding motif of one of the plurality of CPs; wherein the at least one first RNA molecule and the at least one second RNA molecule are encapsulated in the VLP.

In one embodiment, the plurality of CPs is derived from Escherichia virus Qbeta.

In one embodiment, the plurality of CPs is encoded by a nucleotide sequence as denoted by SEQ ID NO: 7.

In one embodiment, the targeting sequence of the second RNA molecule comprises a nucleotide sequence of SEQ ID NO: 4 or SEQ ID NO: 5.

In one embodiment, the targeted site is a nucleotide sequence derived from RNA virus, such as SARS-COV-2, influenza viruses, etc.

In one embodiment, the Cas13 protein recognition sequence comprises a nucleotide sequence of SEQ ID NO: 6.

Accordingly, in the invention, through protecting mRNA encoding a Cas13 protein by the VLP coated on the outer layer, mRNA can stably enter human cells to translate the Cas13 protein, effectively blocking the replication and proliferation of SARS-COV-2, thus treating and preventing COVID-19 caused by SARS-COV-2. The mRNA nanocapsule of the invention can not only overcome the shortcomings of in vitro transcription, but also can completely and safely deliver the mRNA into human cells, thereby producing the target proteins through human cells themselves and achieving the desired effect.

### BRIEF DESCRIPTION OF THE DRAWINGS

FIG. 1 is a schematic of a nucleic acid molecule of a first RNA according to an embodiment of the present invention.

FIG. 2 is a schematic diagram of an mRNA nanocapsule according to an embodiment of the present invention.

FIG. 3 is a schematic of a nucleic acid molecule of a second RNA according to an embodiment of the present invention.

FIG. 4 depicts a composition comprising an mRNA nanocapsule according to an embodiment of the present invention.

FIG. 5 depicts a composition comprising an mRNA nanocapsule according to another embodiment of the present invention.

FIG. 6A to FIG. 6F are an exemplary structure of the VLP according to an embodiment of the present invention.

FIG. 7A and FIG. 7B show experimental results of Test Example 1 of the present invention.

FIG. 8A and FIG. 8B show experimental results of Test Example 2 of the present invention.

FIG. 9A and FIG. 9B show experimental results of Test Example 3 in of the present invention.

FIG. 10A and FIG. 10B show experimental results of Test Example 4 of the present invention.

FIG. 11A and FIG. 11B show experimental results of Test Example 5 of the present invention.

### DETAILED DESCRIPTION OF THE PREFERRED EMBODIMENTS

As used herein, the term “capsid protein” refers to the protein components that self-assemble to form the outer shell or capsid of a virus or virus-like particle, providing structural integrity and protection to the viral genome or encapsulated materials.

As used herein, the term “virus-like particle (VLP)” refers to a non-infectious structure that closely resembles a virus but lacks viral genetic material. VLPs are composed of capsid proteins, which self-assemble into a particle that mimics the morphology and size of viruses. These particles are extensively used in drug delivery due to their ability to encapsulate and protect therapeutic agents, such as nucleic acids or proteins. Examples of VLPs used in drug delivery include those derived from bacteriophages, such as Qβ (Escherichia virus Qbeta) and AP205 (Acinetobacter phage AP205).

As used herein, the term “deoxyribonucleic acid (DNA)” refers to a molecule that encodes the genetic instructions transcribed into RNA (ribonucleic acid), particularly messenger RNA (mRNA), which then serves as a template for protein synthesis.

As used herein, the term “messenger RNA (mRNA)” refers to the type of RNA that carries genetic information transcribed from DNA to the ribosome, where it specifies the amino acid sequence of the protein products of gene expression.

As used herein, the term “CRISPR/Cas system” refers to an adaptive immune system in bacteria. CRISPR/Cas system involves using a Cas protein guided by an RNA molecule to induce specific modifications in the DNA and/or RNA of an organism for genome editing.

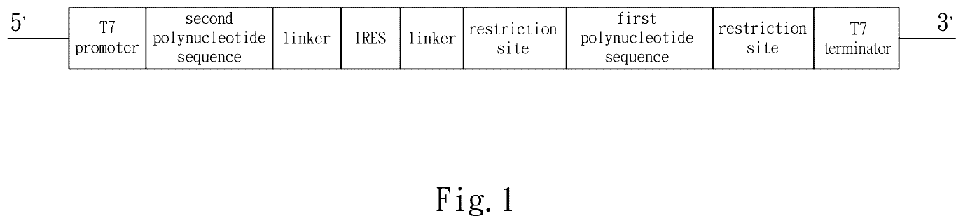

FIG. 1 shows a deoxyribonucleic acid (DNA) molecule encoding an mRNA expressing a Cas13 protein, the Cas13 protein is a nuclease used in the CRISPR/Cas system to cleave specific DNA or RNA. The DNA molecule comprises a first polynucleotide sequence encoding an mRNA expressing a Cas13 protein. In one embodiment, the first polynucleotide sequence includes a nucleotide sequence as denoted by SEQ ID NO: 2 encoding an mRNA expressing a Cas13d protein. In another embodiment, the first polynucleotide sequence includes a nucleotide sequence as denoted by SEQ ID NO: 3 encoding an mRNA expressing a Cas13a protein.

Further, the DNA molecule may also comprise a second polynucleotide sequence encoding an RNA aptamer linking with the mRNA encoded by the first polynucleotide sequence. The RNA aptamer is a short RNA molecule that is designed to bind specifically to a particular target molecule, such as protein, small molecules, or other RNA. The RNA aptamer is designed to recognize a capsid protein. In one embodiment, the second polynucleotide sequence is denoted by SEQ ID NO: 1, encoding an RNA aptamer with a hairpin structure and specifically binding to a capsid protein derived from the Escherichia virus Qbeta (Bacteriophage Qbeta), the capsid protein is encoded from the nucleotide sequence as denoted by SEQ ID NO: 8 (GeneBank accession no. M99039.1).

In one embodiment, the DNA molecule may further comprise an internal ribosome entry site (IRES) between the first polynucleotide sequence and the second polynucleotide sequence.

In one embodiment, the DNA molecule may further comprise two restriction sites located upstream and downstream of the first polynucleotide sequence. The two restriction sites are sequences that can be recognized by any restriction enzyme, such as EcoRI, BamHI, HindIII, XbaI, etc., but are not limited thereto.

In one embodiment, the DNA molecule may further comprise a promoter located at 5′ end and a terminator at 3′ end of the first polynucleotide sequence for the transcription of the RNA polymerase. In an example, T7 promoter and T7 terminator that are recognized by T7 RNA polymerase may be used, but are not limited thereto.

In one embodiment, the DNA molecule may further comprise two linkers located upstream and downstream of the IRES, and the linker may be any polynucleotide sequence of 15 to 30 nucleotides in length. When the nucleic acid molecule is transfected into cells, RNA polymerase recognizes the promoter on the nucleic acid molecule and starts transcription to form the corresponding mRNA which encodes the Cas13 protein.

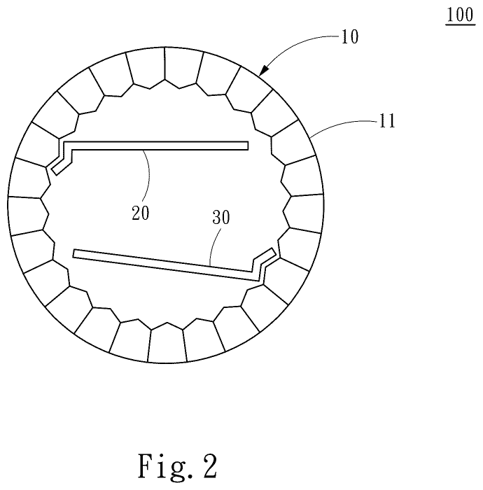

Referring to FIG. 2, it is a schematic diagram of an mRNA nanocapsule 100 according to an embodiment of the present invention. The mRNA nanocapsule 100 is formed by encapsulating the in vivo transcribed RNA in a nanoscale RNA-protein complex structure. The mRNA nanocapsule 100 includes a plurality of capsid proteins (CPs) 11, one or more first RNA molecules 20 and/or one or more second RNA molecules 30. The plurality of capsid proteins 11 self-assemble into a spheroid to form a virus-like particle (VLP) 10. The VLP 10 includes an outer shell and a cavity space. The outer shell may be composed of the plurality of capsid proteins 11 that are self-assembled to form a shell-like structure, such as a protein cage. The cavity space may encapsulate substances, such as drugs or genetic material, for targeted delivery in therapeutic applications. Each of the capsid proteins 11 comprises an RNA-binding motif for recognizing and binding to specific RNA, the RNA-binding motif is located at the surface of the capsid protein 11 facing the cavity space. Each of the one or more first RNA molecules 20 comprises an mRNA encoding a Cas13 protein and a capsid protein binding tag linked with the mRNA. The capsid protein binding tag is an RNA aptamer that recognizes and binds to the RNA-binding motif of one of the capsid proteins 11 so that the one or more first RNA molecule 20s may be encapsidated in the VLP 10.

The nucleotide sequence of each of the one or more second RNA molecules 30 includes a guide RNA, a Cas13 protein recognition sequence, and a VLP recognition sequence. The guide RNA is encoded by a targeting sequence that is reverse and complementary to a targeted site of the virus. The Cas13 protein recognition sequence recognizes the Cas13 protein encoded by the one or more first RNA molecules 20, allowing the Cas13 protein to cleave the targeted site of the virus.

The ratio of the number of moles of the one or more first RNA molecules 20 to the one or more second RNA molecules 30 is determined according to the amount and type of viruses infected or different VLPs, thereby achieving an optimized antiviral effect. In one embodiment, the number of moles of the one or more first RNA molecules 20 is less than or equal to that of the one or more second RNA molecules 30. For example, the ratio of the number of moles of the one or more first RNA molecules 20 to the one or more second RNA molecules 30 is 1:5, but is not limited thereto. According to one embodiment of the first RNA molecule 20, the mRNA is encoded by a nucleotide sequence as denoted by SEQ ID NO: 2, and the mRNA expresses a Cas13d protein. In another embodiment, the mRNA is encoded by a nucleotide sequence as denoted by SEQ ID NO: 3, and the mRNA expresses a Cas13a protein.

In one embodiment, the nucleotide sequence encoding the one or more first RNA molecules 20 may further comprise an internal ribosome entry site (IRES) between the first polynucleotide sequence and the second polynucleotide sequence.

In one embodiment, the nucleotide sequence encoding the one or more first RNA molecules 20 may further comprise two restriction sites located upstream and downstream of the first polynucleotide sequence. The two restriction sites are sequences that can be recognized by any restriction enzyme, such as EcoRI, BamHI, HindIII, XbaI, etc., but are not limited thereto.

In one embodiment, the nucleotide sequence encoding the one or more first RNA molecule 20 may further comprise a promoter located at 5′ end and a terminator at 3′ end of the first polynucleotide sequence for the transcription of the RNA polymerase. In a non-limiting example, T7 promoter and T7 terminator that are recognized by T7 RNA polymerase may be used.

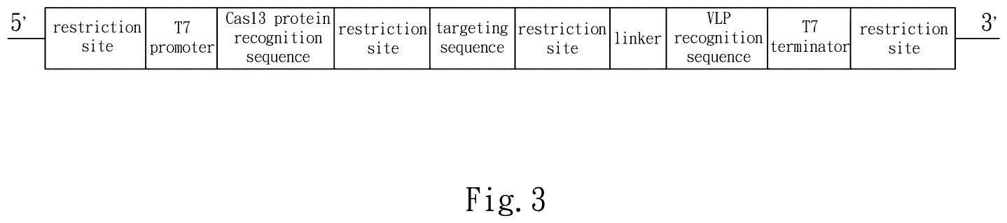

FIG. 3 is a schematic of a nucleotide sequence encoding the one or more second RNA molecules 30, comprising the Cas13 protein recognition sequence, the targeting sequence, and a VLP recognition sequence from the 5′ end to the 3′ end. The VLP recognition sequence encodes the RNA sequence of the capsid protein binding tag.

In one embodiment, the targeted site is the viral genome, and the targeting sequence is reverse and complementary to a specific segment in the RNA sequence of the virus. In one embodiment, the length of the targeting sequence ranges between 20 and 40 base pairs, but is not limited thereto. In one embodiment, the targeting sequence may include a nucleotide sequence as denoted by SEQ ID NO: 4; in other embodiments, the targeting sequence may include a nucleotide sequence as denoted by SEQ ID NO: 5. In one embodiment, the targeted site may be a nucleotide sequence derived from SARS-COV-2.

The Cas13 protein recognition sequence may be used to bind to a specific region of the Cas13 protein and guide the Cas13 protein to the targeted site to cleave the virus RNA. In one embodiment, the Cas13 protein recognition sequence may be an RNA hairpin structure transcribed from the nucleotide sequence as denoted by SEQ ID NO: 6.

In one embodiment, the second RNA molecule 30 further includes a promoter at the upstream of the Cas13 protein recognition sequence, and a terminator at the downstream of the VLP recognition sequence. In a non-limiting example, T7 promoter and T7 terminator that are recognized by T7 RNA polymerase may be used.

In one embodiment, the second RNA molecule 30 further includes a first pair of restriction sites located at two ends of the second RNA molecule 30, and a second pair of restriction sites located at the upstream and downstream of the targeting sequence. The two pairs of restriction sites may be the same or different, and can be recognized by any restriction enzymes, such as EcoRI, BamHI, HindIII, XbaI, etc., but are not limited thereto.

In one embodiment, the second RNA molecule 30 further includes a linker located upstream of the VLP recognition sequence, and the linker is any polynucleotide sequence of 15 to 30 nucleotides in length.

In a non-limiting example, the capsid proteins 11 may be derived from Escherichia virus Qbeta. The capsid proteins 11 may be encoded by a nucleotide sequence as denoted by SEQ ID NO: 7 (GeneBank accession no. M99039.1), and comprise an amino acid sequence as denoted by SEQ ID NO:8. In one embodiment, the VLP 10 with a diameter of 30 nanometers may be obtained from self-assembly of a number of one hundred and eighty (180) of the capsid proteins 11. Each of the capsid proteins 11 may comprise an RNA-binding motif determined from the amino acid residues at position 59 to position 68 of the SEQ ID NO:8, as denoted by SEQ ID NO: 15.

The capsid protein binding tag of the one or more first RNA molecule 20 and the one or more second RNA molecule 30 are designed to recognize and bind the at least one of the capsid proteins 11, and the capsid protein binding tag is encoded by a VLP recognition sequence as denoted by SEQ ID NO: 1. The capsid protein binding tag may be formed as a hairpin structure to recognize and bind to the RNA-binding motif as denoted by SEQ ID NO: 15 of one of the capsid proteins 11. The capsid protein binding tag may form specific molecular interactions to the amino acid residues at positions 1-4, 6, 8, 10 of the SEQ ID NO: 15.

In addition to the capsid proteins derived from Escherichia virus Qbeta, other capsid proteins capable of forming VLPs for transportation may also be used for generating the mRNA nanocapsule of the invention. The potential candidates may be selected from the capsid proteins generated by the single-stranded RNA bacterial phage based on their crystal structures, and the priority is given to the capsid proteins that form a dimer conformation candidate. Then, the potential candidates are compared to the capsid proteins derived from Escherichia virus Qbeta through comparative approach and/or structural superimposition in silico, and the assembly ability and molecular interaction with the capsid protein binding tag are tested through the standard methods in molecular biology in vitro. For example, the capsid proteins derived from Acinetobacter phage AP205 can also be used to form the mRNA nanocapsule of the invention. The nucleotide sequence encoding the capsid protein of Acinetobacter phage AP205 is denoted by SEQ ID NO: 9, and an amino acid sequence of the capsid protein is denoted by SEQ ID NO: 10.

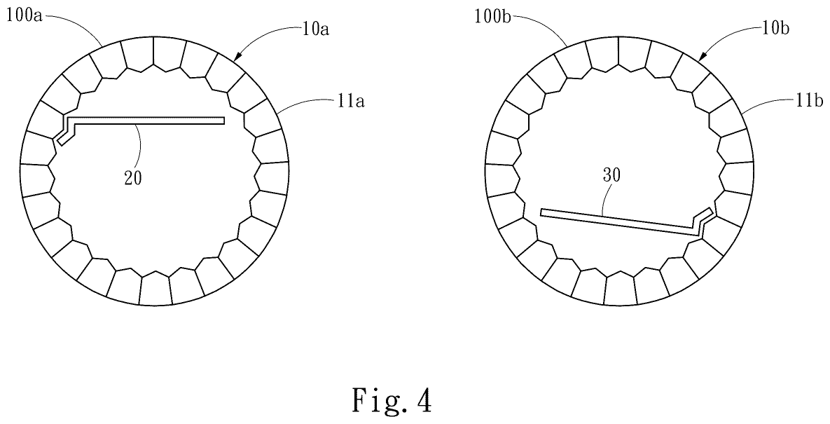

FIG. 4 depicts composition comprising an mRNA nanocapsule according to an embodiment of the present invention. The composition includes a plurality of mRNA nanocapsules 100a and a plurality of guide RNA nanocapsules 100b. Each of the plurality of mRNA nanocapsules 100a includes a first VLP 10a and one or more mRNA 20 encoding Cas13 protein. The first VLP 10a is formed by self-assembly of a plurality of first CPs 11a. Each of the guide RNA nanocapsules 100b includes a second VLP 10b and one or more guide RNA 30, and the second VLP 10b is formed by self-assembly of a plurality of second CPs 11b. The structures of the mRNA 20 and the guide RNA 30 are the same as those in the previous embodiment, and will not be described again herein. The first CPs 11a and the second CPs 11b may be the same or different. The ratio of the number of moles of the mRNA nanocapsules 100a to the guide RNA nanocapsules 100b is determined according to the amount and type of viruses infected or different VLPs, thereby achieving an optimized antiviral effect. In one embodiment, the ratio of the number of moles of the mRNA nanocapsules 100a to the guide RNA nanocapsules 100b is between 1:10 and 1:30, for example, the ratio of the number of moles is 1:20, but is not limited thereto.

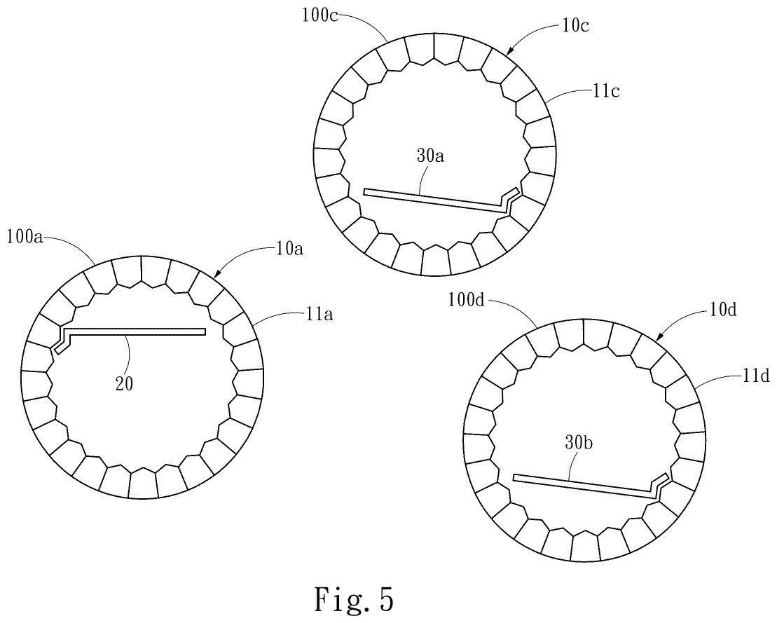

FIG. 5 depicts a composition comprising an mRNA nanocapsule according to another embodiment of the present invention. The composition includes a plurality of mRNA nanocapsules 100a, a plurality of first guide RNA nanocapsules 100c, and a plurality of second guide RNA nanocapsules 100d. Each of the mRNA nanocapsules 100a includes a first VLP 10a and one or more mRNA 20 encoding Cas13 protein. The first VLP 10a is formed by self-assembly of a plurality of first CPs 11a. Each of the first guide RNA nanocapsules 100c includes a third VLP 10c and one or more first guide RNA 30a, and the third VLP 10c is formed by self-assembly of a plurality of third CPs 11c. Each of the second guide RNA nanocapsules 100d includes a fourth VLP 10d and one or more second guide RNA 30b, and the fourth VLP 10d is formed by self-assembly of a plurality of fourth CPs 11d. The first guide RNA 30a and the second guide RNA 30b respectively comprise targeting sequences with different nucleotide sequences. In one embodiment, the targeting sequence in the first guide RNA 30a includes a nucleotide sequence of SEQ ID NO: 4, and the targeting sequence in the second guide RNA 30b includes a nucleotide sequence of SEQ ID NO: 5. The first CPs 11a, the third CPs 11c, and the fourth CPs 11d may be the same or different. The structures of the mRNA 20, the first guide RNA 30a, and the second guide RNA 30b are the same as those in the foregoing embodiments, and will not be described again herein.

The invention further provides a use of the mRNA nanocapsule in preparing a drug for treating or preventing SARS-COV-2. When the mRNA nanocapsule 100 enters the SARS-COV-2 infected cell, the Cas13 protein is translated, and the targeted site derived from the nucleotide sequence of SARS-COV-2 is bound with the guide RNA since the targeted site is complementary to the targeting sequence of the guide RNA, thereby guiding the Cas13 protein to cleave the targeted site.

The following examples are only used to illustrate the purpose of the invention but not limit the scope of the invention. Those skilled in the art can produce other specific embodiments, substitutions and changes according to the disclosure and teachings of the present invention.

### [Example 1] Preparation of Target Vector

An RNA segment of the SARS-COV-2 gene was inserted into a green fluorescent protein (GFP) expression plasmid as the target vector to be cleaved in the present invention.

### [Example 2] Preparation of Capsule Vector

A nucleotide encoding the capsid proteins from the source of Escherichia virus Qbeta (GeneBank accession no. M99039.1) was inserted into a plasmid as a capsule vector for the production of VLP.

### [Example 3] Preparation of Cas Vector

A nucleotide identifying the VLP and a nucleotide encoding the Cas13 protein were inserted into a plasmid as a Cas vector for cleaving the target vector of Example 1. The nucleotide sequence of the Cas vector can be denoted by SEQ ID NO: 11 for encoding Cas13d protein or SEQ ID NO: 12 for encoding Cas13a protein.

### [Example 4] Preparation of Guide RNA Vector

A nucleotide identifying the VLP and a nucleotide encoding the guide RNA were inserted into a plasmid as a guide RNA vector for identifying the target vector of Example 1. The nucleotide sequence of the guide RNA vector is denoted by SEQ ID NO: 13 or SEQ ID NO: 14.

### [Example 5] Preparation of Nanocapsules

The capsule vector of Example 2, the Cas vector of Example 3, and the guide RNA vector of Example 4 were co-transformed into Escherichia coli so that the translation of capsid protein and the transcription of Cas13 mRNA or guide RNA were carried out simultaneously in Escherichia coli. Cas13 mRNA and guide RNA bound the VLP formed by self-assembly of capsid proteins through the nucleotide identifying the VLP, so as to spontaneously assemble into nanocapsules.

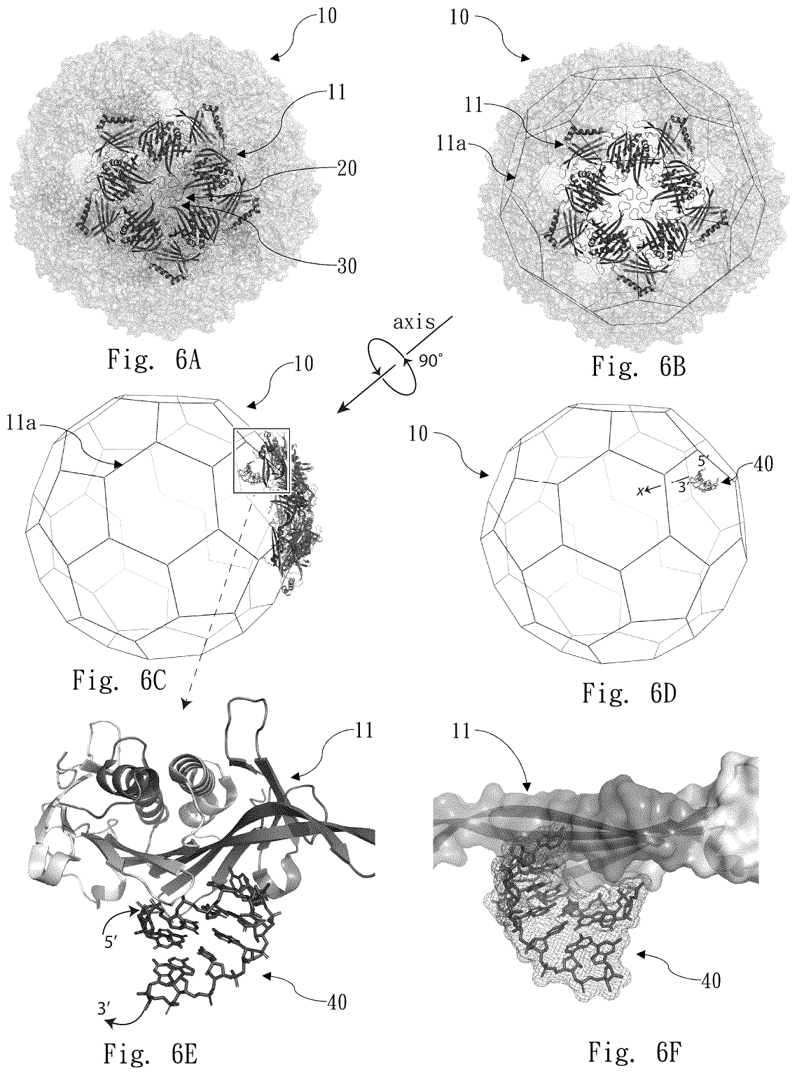

FIG. 6A to FIG. 6G are an exemplary structure of the VLP according to an embodiment of the present invention. FIG. 6A depicts a non-limiting exemplary model structure of the VLP 10 formed by 180 units of the capsid proteins 11. The VLP 10 includes the capsid proteins 11, the first RNA molecule 20, and the second RNA molecule 30. The first RNA molecule 20 and the second RNA molecule 30 are shown in a dark grey area packed within the VLP 10. The VLP 10 is a hollow spheroid composed of hexagonal units assembled from several units of the capsid proteins 11. FIG. 6B depicts a non-limiting exemplary model structure by showing a skeleton structure 11a formed by the capsid proteins 11 and omitting the first RNA molecule 20 and the second RNA molecule 30 in FIG. 6A.

FIG. 6C depicts a non-limiting exemplary model structure of the VLP 10, which is obtained by rotating an axis by 90 degrees from FIG. 6B. The VLP 10 in FIG. 6C shows the capsid protein binding tag 40 binds to the RNA-binding motif of the capsid proteins 11. FIG. 6D depicts a non-limiting exemplary model structure of the VLP 10 by removing the capsid proteins 11 in FIG. 6C, which shows the hairpin structure of the capsid protein binding tag 40 with the 5′ end and the 3′ end. The “x” in FIG. 6D may be an RNA sequence, such as Cas13 mRNA and guide RNA, covalently linking with the hairpin structure.

FIG. 6E is an enlarged view of a portion of the VLP 10 in FIG. 6C, showing a dimer structure assembled by two identical units of the capsid proteins 11 and the capsid protein binding tag 40. The RNA-binding motifs of the dimer structure of the capsid proteins 11 are bound with the capsid protein binding tag 40, which may be covalently connected to the mRNA encoding a Cas13 protein that forms the first RNA molecule 20 or to the guide RNA encoded by a targeting sequence. In this example, the capsid protein binding tag 40 is a hairpin structure binding with the dimer structure of the two capsid proteins 11 but is not limited thereto. FIG. 6F shows the direct contact region of the RNA-binding motifs where the RNA hairpin tag 40 (stick presentation in the mesh of the van der Waals) is in direct contact with the binding pocket formed by the beta-sheet regions (cartoon presentation in the surface of the van der Waals) of the dimer structure of the two capsid proteins 11. It shows a three-dimensional structure of the connection between the RNA-binding motifs of the capsid proteins 11 and the capsid protein binding tag 40.

### [Test Example 1] Therapeutic Effect of mRNA Nanocapsules on COVID

The target vector of Example 1 was transfected into human embryonic kidney cells (HEK293), and the untransfected vector was washed away with PBS buffer. Then, the capsule vector of Example 2, the Cas vector of Example 3 as denoted by SEQ ID NO: 11 for encoding Cas13d protein, and the guide RNA vector as denoted by SEQ ID NO: 13 of Example 4 were co-transformed into Escherichia coli to generate the nanocapsules as Example 5, and the nanocapsules were added to HEK293 cells so that the mRNA and the guide RNA in the nanocapsules were transfected into HEK293 cells. The transfected cells were cultured for 4 hours, 10 hours, and 21 hours. Fluorescence images were captured by a fluorescence microscope, and the fluorescence values were analyzed by image analysis software.

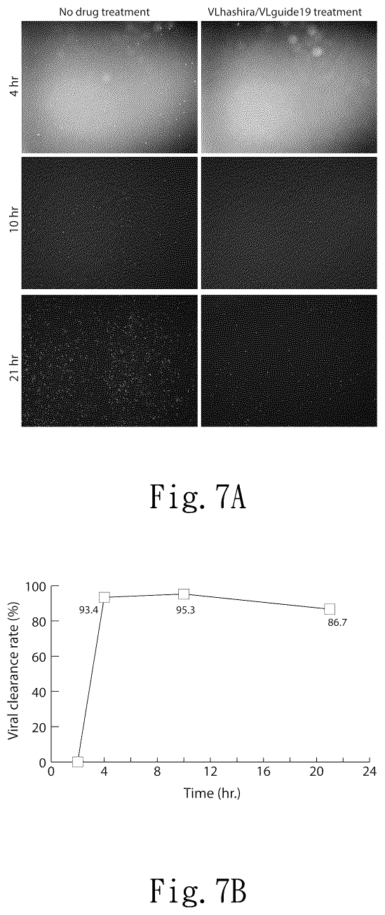

FIG. 7A and FIG. 7B showed the experimental results of this test example. FIG. 7A, the left column showed the fluorescence image of the cells of the control group that were not treated with the mRNA nanocapsules. The right column showed the fluorescence image of the cells of the experimental group treated with the mRNA nanocapsules. After 21 hours of culture, the amount of fluorescence of the cells in the experimental group was significantly lower than that of the control group, i.e., the mRNA nanocapsules significantly reduced the amount of the target vector, which comprises the RNA segment of SARS-COV-2 gene in the cells. The fluorescence values were analyzed by the image analysis software, and the viral clearance rates were calculated, as shown in FIG. 7B, after 10 hours of culture, the viral clearance rate of the experimental group treated with the mRNA nanocapsules was greater than 90%, and after 21 hours of culture, the viral clearance rate could still maintain at 86.7%. The experimental results showed that the mRNA nanocapsules of the invention were therapeutically effective to COVID-19 caused by SARS-COV-2.

### [Test Example 2] Preventive Effect of Multi-Dose mRNA Nanocapsules on SARS-COV-2

The nanocapsules of Example 5 were first added to HEK293 cells in multiple doses, so that the mRNA and the guide RNA in the nanocapsules were transfected into HEK293 cells, and the untransfected nanocapsules was removed; then, the target vector of Example 1 was transfected into HEK293 cells, and the untransfected vector was washed away with PBS buffer.

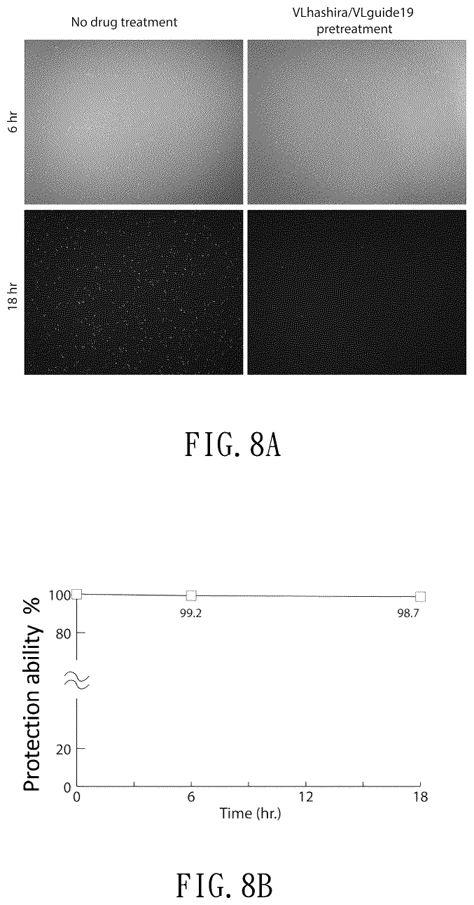

FIG. 8A and FIG. 8B showed the experimental results of this test example. FIG. 8A, the left column showed the fluorescence image of the cells of the control group that were not treated with the mRNA nanocapsules. The right column showed the fluorescence image of the cells of the experimental group treated with the mRNA nanocapsules. After 18 hours of culture, the amount of fluorescence of the cells in the experimental group was significantly lower than that of the control group, as shown in FIG. 8B, the protection ability of the experimental group with pre-treated mRNA nanocapsules was still close to 100% after 18 hours. Thus, according to the experimental results, the mRNA nanocapsules pre-treated to the cells effectively prevented COVID-19 caused by SARS-COV-2.

### [Test Example 3] Preventive Effect of Single-Dose mRNA Nanocapsule on SARS-COV-2

The nanocapsules of Example 5 were first added to HEK293 cells in a single dose so that the mRNA and guide RNA in the nanocapsules were transfected into HEK293 cells, and the untransfected nanocapsules was removed; then, the target vector of Example 1 was transfected into HEK293 cells, and the untransfected vector was washed away with PBS buffer.

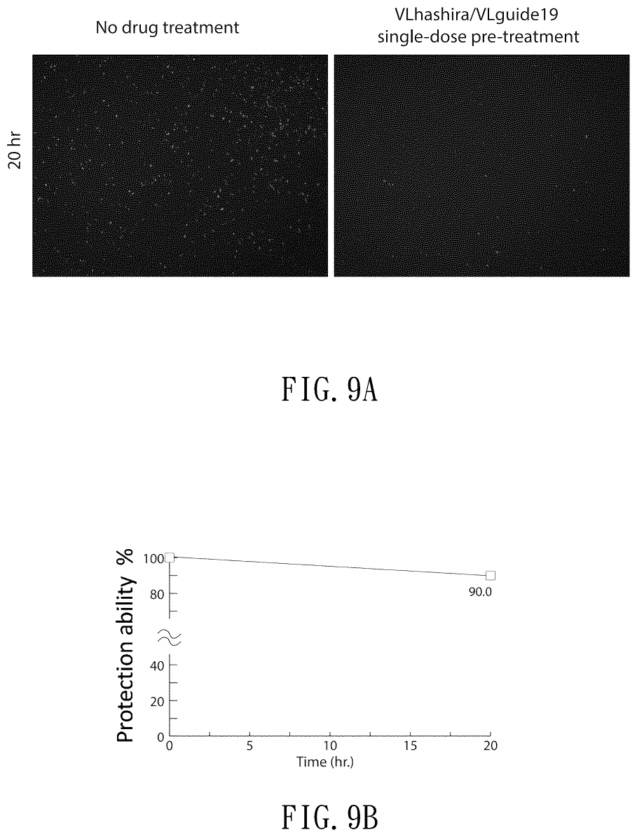

FIG. 9A and FIG. 9B showed the experimental results of this test example. FIG. 9A, the left column showed the fluorescence image of the cells of the control group that were not treated with the nanocapsules. The right column showed the fluorescence image of the cells of the experimental group treated with the nanocapsules. After 20 hours of culture, the amount of fluorescence of the cells in the experimental group was significantly lower than that of the control group, as shown in FIG. 9B, the protection ability in the experimental group with pre-treated mRNA nanocapsules was still close to 90% after 20 hours. Thus, according to the experimental results, the mRNA nanocapsules pre-treated to the cells effectively prevented COVID-19 caused by SARS-COV-2.

### [Test Example 4] mRNA Nanocapsule could Quickly Adapt to Viral RNA Mutations

In this test example, natural GFP expression plasmids without RNA segment of SARS-COV-2 gene were transfected into HEK293 cells as mutations of SARS-COV-2 RNA. Then, the nanocapsules of the invention were added to HEK293 cells. The difference from the above test examples lies in the arrangement that the targeting sequence of the guide RNA used in this test example was reverse and complementary to the nucleotide sequence of the natural GFP expression plasmid.

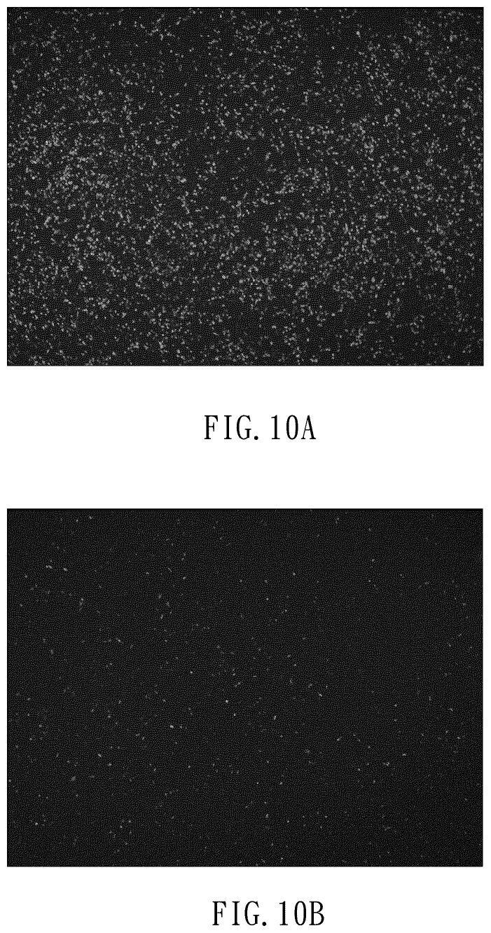

FIG. 10A and FIG. 10B showed the experimental results of this test example. FIG. 10A showed the fluorescence image of the cells of the control group that were not treated with the mRNA nanocapsules. FIG. 10B showed the fluorescence image of the cells of the experimental group treated with the mRNA nanocapsules.

Comparing FIG. 10A with FIG. 10B, the amount of fluorescence of the cells in the experimental group was significantly lower than that of the control group, indicating that the mRNA nanocapsules of the invention could quickly adapt to the infection of viral RNA mutations.

### [Test Example 5] Specificity of mRNA Nanocapsules

In this test example, the natural GFP expression plasmids were transfected into HEK293 cells, and then the nanocapsules of the invention were added into HEK293 cells, the targeting sequence of the guide RNA was the reverse and complementary to SARS-COV-2 (not the natural GFP expression plasmid).

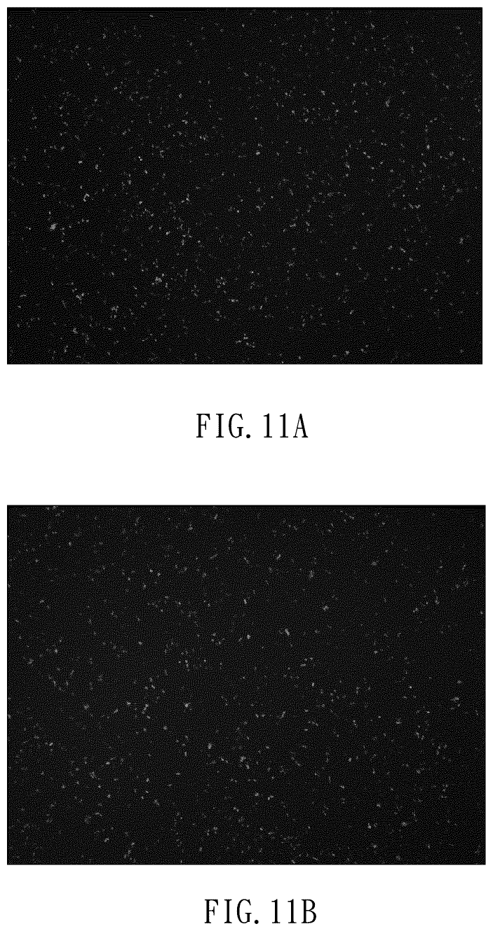

FIG. 11A and FIG. 11B showed the experimental results of this test example. FIG. 11A showed the fluorescence image of the cells of the control group that were not treated with the mRNA nanocapsules. FIG. 11B showed the fluorescence image of the cells of the experimental group treated with the mRNA nanocapsules.

Comparing FIG. 11A with FIG. 11B, the amount of fluorescence of the cells in the experimental group was close to that of the control group, indicating that the guide RNA comprising the targeting sequence that was reverse and complementary to SARS-COV-2 could not recognize the natural GFP expression plasmids, thus could not guide the Cas13 protein to cleave the natural GFP expression plasmids. The experimental results showed that the mRNA nanocapsules of the invention have specificity through the designed targeting sequence.

What is claimed is:

1. An mRNA nanocapsule, comprising:
a plurality of capsid proteins, self-assembling into a virus-like particle, wherein each of the capsid proteins comprises an RNA-binding motif;
one or more first RNA molecules, comprising an mRNA encoding a Cas13 protein and a capsid protein binding tag, wherein the capsid protein binding tag recognizes and binds to the RNA-binding motif of one of the capsid proteins; and
one or more second RNA molecules, encoded by a nucleotide sequence comprising a targeting ta sequence reverse and complementary to a targeted site, a Cas13 protein recognition sequence, and the capsid protein binding tag recognizing and binding to the RNA-binding motif of one of the capsid proteins;
wherein the capsid protein binding tag is encoded by a virus-like particle recognition sequence as denoted by SEQ ID NO: 1, and the RNA-binding motif comprises an amino acid sequence as denoted by SEQ ID NO: 15; and
wherein the one or more first RNA molecules and the one or more second RNA molecules are encapsulated in the virus-like particle.

2. The mRNA nanocapsule according to claim 1, wherein the capsid proteins are derived from the Escherichia virus Qbeta.

3. The mRNA nanocapsule according to claim 2, wherein the capsid proteins are encoded by a nucleotide sequence as denoted by SEQ ID NO: 7.

4. The mRNA nanocapsule according to claim 1, wherein the targeting sequence of the one or more second RNA molecules comprises a nucleotide sequence of SEQ ID NO: 4 or SEQ ID NO: 5.

5. The mRNA nanocapsule according to claim 1, wherein the targeted site is a nucleotide sequence derived from RNA virus.

6. The mRNA nanocapsule according to claim 1, wherein the Cas13 protein recognition sequence comprises a nucleotide sequence of SEQ ID NO: 6.

7. The mRNA nanocapsule according to claim 1, wherein the number of moles of the one or more first RNA molecules is less than or equal to that of the one or more second RNA molecules.
